(19) United States
(12) Patent Application Publication
(10) Pub. No.: US 2020/0283352 A1
(43) Pub. Date: Sep. 10, 2020

(54) PROCESS AND PLANT FOR PRODUCING OLEFINS FROM OXYGENATES

(71) Applicant: L'Air Liquide, Societe Anonyme pour l'Etude et l'Exploitation des Procedes Georges Claude, Paris (FR)

(72) Inventors: Lutz JANKO, Frankfurt am Main (DE); Martin GORNY, Frankfurt am Main (DE); Stephane HAAG, Frankfurt (DE); Bernd AHLERS, Frankfurt am Main (DE)

(73) Assignee: L'Air Liquide, Societe Anonyme pour l'Etude et l'Exploitation des Procedes Georges Claude, Paris (FR)

(21) Appl. No.: 16/810,315

(22) Filed: Mar. 5, 2020

(30) Foreign Application Priority Data
Mar. 6, 2019 (EP) EP 19020106.1

(51) Int. Cl. C07C 1/22; C07C 41/09; B01J 19/24; B01D 3/14

(52) U.S. Cl. CPC C07C 1/22; C07C 41/09; B01J 2219/0004; B01D 3/143; B01J 19/245

(57) ABSTRACT

The invention relates to a process and a plant for producing an olefins-containing hydrocarbon product by reaction of an oxygenates-containing reactant mixture, which is divided into a plurality of reactant mixture substreams, in a multi-stage oxygenate-to-olefin (OTO) synthesis reactor comprising a plurality of serially connected reaction sections comprising catalyst zones, wherein a feeding apparatus for a reactant mixture substream is arranged upstream of each catalyst zone. In each of these reaction sections a reactant mixture substream is introduced and therein under oxygenates conversion conditions converted into olefins and further hydrocarbons, wherein all reaction sections save for the first are additionally supplied with the product stream from the respective upstream reaction section. In addition at least one steam stream is introduced into at least one reaction section and at least one hydrocarbons-containing recycle stream is introduced into at least one reaction section. The OTO synthesis reactor product is fractionated in a multi-stage workup apparatus to obtain a plurality of hydrocarbon product fractions of which at least one is recycled to the OTO synthesis reactor as a recycle stream. According to the invention all reactant mixture substreams, steam streams and recycle streams are introduced into the OTO synthesis reactor in gaseous/vaporous form.

### CROSS REFERENCE TO RELATED APPLICATION

This application claims the benefit of priority under 35 U.S.C. § 119 (a) and (b) to European patent application No. EP19020106.1, filed Mar. 6, 2019, the entire contents of which are incorporated herein by reference.

### FIELD OF THE INVENTION

The invention relates to a process for producing an olefins-containing hydrocarbon product, comprising in particular ethylene and propylene, by reaction of an oxygenates-containing reactant mixture, which is divided into a plurality of reactant mixture substreams, in a multi-stage oxygenate-to-olefin (OTO) synthesis reactor comprising a plurality of serially connected reaction sections which each contain catalyst zones comprising solid catalysts active and selective for OTO synthesis, wherein a feeding apparatus for a reactant mixture substream is arranged upstream of each catalyst zone. In each of these reaction sections a reactant mixture substream is introduced and therein under oxygenates conversion conditions converted into olefins and further hydrocarbons, wherein all reaction sections save for the first are additionally supplied with the product stream from the respective upstream reaction section. In addition at least one steam stream is introduced into at least one reaction section and at least one hydrocarbons-containing recycle stream is introduced into at least one reaction section. The OTO synthesis reactor product is fractionated in a multi-stage workup apparatus to obtain a plurality of hydrocarbons-containing hydrocarbon product fractions of which at least one is recycled to the OTO synthesis reactor as a recycle stream.

The invention further relates to a plant for performing such a process.

### BACKGROUND OF THE INVENTION

Short-chain olefins, especially propylene (propene), are among the most important commodities in the chemical industry. The reason for this is that, proceeding from these unsaturated compounds with a short chain length, it is possible to form molecules having a long-chain carbon skeleton and additional functionalizations.

The main source of short-chain olefins in the past was steam cracking, i.e. thermal cracking in mineral oil processing. In the past few years, however, further processes for preparing short-chain olefins have been developed. One reason for this is rising demand that can no longer be covered by the available sources; secondly, the increasing scarcity of fossil raw materials is requiring the use of different starting materials.

The so-called MTP (methanol-to-propylene) or else MTO (methanol-to-olefin) processes for preparing propylene and other short-chain olefins proceed from methanol as starting material. In this connection reference is also generally made to oxygenate-to-olefin (OTO) processes, since oxygen-containing organic components such as methanol or dimethyl ether (DME) are also referred to as oxygenates. These heterogeneously catalysed processes thus initially partly convert methanol into the intermediate dimethyl ether and subsequently from a mixture of methanol and dimethyl ether form a mixture of ethylene and propylene and hydrocarbons having a higher molar mass, especially including olefins. Also present in the product stream is water which derives from the process steam optionally supplied to the OTO reactor for reaction modulation and the reaction water produced in the OTO reactor.

In the MTP process known from the prior art pure methanol initially obtained from crude methanol by distillation is the starting material for the reaction. Since the hydrocarbon synthesis starting from methanol in the OTO reactor is strongly exothermic, initially in an etherification reactor arranged upstream of the OTO reactor, the so-called DME reactor, pure methanol is vaporized and supplied and therein, after optional addition of steam as diluent, converted into dimethyl ether (DME) and water in straight pass by heterogeneously catalysed dehydration. The resulting product mixture contains not only DME but also unconverted methanol and water; after discharging from the DME reactor it is typically cooled and partially condensed to obtain a DME-rich gas phase and a water- and methanol-rich liquid phase, both of which are employed as the reactant mixture for the subsequent OTO reaction.

The subsequent conversion of the pre-reacted input mixture containing DME and methanol as oxygenates in a multi-stage OTO reactor is taught for example in published European patent EP 2032245 B1. The OTO reactor comprises a plurality of reaction sections traversed from top to bottom by the oxygenates-containing input mixture and arranged inside a closed, upright container, each composed of a supporting tray having disposed thereupon a catalyst zone formed from a dumped bed of granular molecular sieve catalyst, for example of the structure type ZSM-5. In an intermediate space delimited in the upward and downward directions in each case by two adjacent reactions sections is an atomizer system in the form of a group of jet tubes having two-fluid nozzles which is used for uniform spraying of the water- and methanol-rich liquid phase obtained from the DME reactor using the DME-rich gas phase obtained from the DME reactor as propellant. In addition to the thus-achieved fine distribution of the reactant mixture this has the further advantage that the vaporization enthalpy required for vaporization of the fine liquid droplets is withdrawn from the reaction sections and in particular the catalysts zones and thus ensures cooling thereof so that the strong evolution of heat from the OTO reaction is readily controllable. Said evolution of heat would otherwise cause the reaction temperature and thus the process conditions in the OTO reactor to be subject to strong local variations with the result that optimal process management would not be achievable even within a reaction section let alone over the entire OTO reactor. This would cause a reduction in the conversion and/or the selectivity, and consequently also the yield, of valuable target products such as a short chain olefins. Severe local heating especially also in the catalyst zones, so-called hotspots, and consequent catalyst damage, premature catalyst deactivation and a resulting reduction in selectivity for the desired product would also ensue.

However, one disadvantage of the reaction management taught in EP 2032245 B1 is that the employed vapour/liquid distribution system for feeding the oxygenate-containing reactant mixture to the reaction zones is a complex and costly system. It comprises many instruments, pipes, compensators and nozzles and is therefore sensitive to operator error. Correct operation therefore requires significant proficiency and training input so that the operating team can safely master startup and shutdown of the plant and the switching of the operating mode from normal operation to regeneration of the catalyst.

Safe and outage-free operation further requires a high pressure drop over the entire system for the atomization and uniform distribution at different flow rates of the reactant mixture. This limits the operating flexibility of the system. Furthermore, the costs for upstream equipment parts become unnecessarily high since due to the comparatively high pressure drop over the employed vapour/liquid distribution system the pressure level in the DME reactor upstream of the OTO reactor and the supplying equipment parts is likewise relatively high, thus increasing wall thicknesses and costs.

European patent application EP 2760809 A1 discloses a process in which hydrocarbons-containing recycle gas is mixed with purified dimethyl ether and steam and subsequently applied to the reaction sections of a multi-stage OTO reactor. The high purity of the dimethyl ether and the associated switchover to pure gas feeding makes it possible to eschew an addition of water and/or oxygenates in liquid form for cooling. However, the purification of the dimethyl ether by removal of the unconverted methanol and the water formed by the DME formation reaction is very laborious. Furthermore, very high purities of the employed dimethyl ether are required, thus further increasing the energy demand of the process.

### SUMMARY OF THE INVENTION

It must therefore further be noted that there remains a need for a simple, robust process for producing olefins by conversion of an oxygenates-containing reactant mixture in a multi-stage oxygenate-to-olefin (OTO) synthesis reactor with a low energy demand. The invention accordingly has for its object to provide such a process and a corresponding plant.

This object is achieved essentially by a process having the features of claim 1. Further, especially preferred, embodiments of the process according to the invention may be found in the dependent claims.

Process According to an Embodiment of the Invention:

Process for producing an olefins-containing hydrocarbon product comprising ethylene and propylene by conversion of an oxygenates-containing reactant mixture, which is divided into a plurality of reactant mixture substreams, in a multi-stage oxygenate-to-olefin (OTO) synthesis reactor, comprising the following steps:

(a) providing the multistage OTO synthesis reactor having a plurality of serially connected reaction sections in fluid connection with one another comprising a first reaction section and at least one subsequent reaction section which each contain catalyst zones comprising solid catalysts that are active and selective for OTO synthesis, wherein upstream of each catalyst zone a feeding apparatus for a reactant mixture substream is arranged and wherein the last reaction section in the direction of flow is in fluid connection with a conduit for discharging an OTO synthesis reactor product,

(b) introducing a reactant mixture substream into each reaction section via the respective feeding apparatus, wherein the at least one subsequent reaction section is additionally supplied with the product stream from the respective upstream reaction section, introducing at least one steam stream into at least one reaction section, introducing at least one recycle stream into at least one reaction section,

(c) at least partially converting the supplied oxygenates in the catalyst zones under oxygenate conversion conditions into olefins and further hydrocarbons, discharging the OTO synthesis reactor product,

(d) separating the OTO synthesis reactor product in a multistage workup apparatus operating by means of thermal separation processes to obtain a plurality of hydrocarbons-containing hydrocarbon product fractions,

(e) discharging an olefins-containing, in particular ethylene- and/or propylene-containing, hydrocarbon product from the workup apparatus,

(f) recycling at least a portion of one or more hydrocarbon product fractions to the OTO synthesis reactor as a recycle stream or recycle streams and introducing the recycle stream(s) into at least one reaction section,

characterized in that all reactant mixture substreams, steam streams and recycle streams are introduced into the OTO synthesis reactor exclusively in gaseous/vaporous form.

Plant According to the Invention:

Plant for producing an olefins-containing hydrocarbon product comprising ethylene and propylene by conversion of an oxygenates-containing reactant mixture, which is divided into a plurality of reactant mixture substreams, in a multi-stage oxygenate-to-olefin (OTO) synthesis reactor comprising the following constituents:

(a) a multistage OTO synthesis reactor having a plurality of serially connected reaction sections in fluid connection with one another comprising a first reaction section and at least one subsequent reaction section which each contain catalyst zones comprising solid catalysts that are active and selective for OTO synthesis, wherein upstream of each catalyst zone a feeding apparatus for a reactant mixture substream is arranged and wherein the last reaction section in the direction of flow is in fluid connection with a conduit for discharging an OTO synthesis reactor product,

(b) means for introducing a reactant mixture substream into each reaction section via the respective feeding apparatus, means for introducing at least one steam stream into at least one reaction section, means for introducing at least one recycle stream into at least one reaction section,

(c) means for adjusting oxygenate conversion conditions, means for discharging the OTO synthesis reactor product,

(d) a multi-stage workup apparatus operating by means of thermal separation processes and suitable for separating the OTO synthesis reactor product into a plurality of hydrocarbons-containing hydrocarbon product fractions, means for introducing the OTO synthesis reactor product into the workup apparatus,

(e) means for discharging an olefins-containing, in particular ethylene- and/or propylene-containing, hydrocarbon product from the workup apparatus,

(f) means for recycling at least a portion of one or more hydrocarbon product fractions obtained in the workup apparatus to the OTO synthesis reactor as a recycle stream or recycle streams and means for introducing the recycle stream(s) into at least one reaction section, characterized in that all means recited under (b) are configured such that all reactant mixture substreams, steam streams and recycle streams are introduceable into the OTO synthesis reactor in gaseous/vaporous form.

The oxygenate conversion conditions required for the conversion of oxygenates to olefin products are known to the person skilled in the art from the prior art, for example the publications discussed in the introduction. These are those physicochemical conditions under which a measurable conversion, preferably one of industrial relevance, of oxygenates to olefins is achieved. Necessary adjustments of these conditions to the respective operational requirements will be made on the basis of routine experiments. Any specific reaction conditions disclosed may serve here as a guide, but they should not be regarded as limiting in relation to the scope of the invention.

Thermal separation processes for the purposes of the invention include all separation processes based on the establishment of a thermodynamic phase equilibrium. Distillation or rectification are preferred. In principle, however, the use of other thermal separation processes is also conceivable, for example of extraction or extractive distillation.

In the context of the present invention a purification step is to be understood as meaning in principle all process steps that make use of a thermal separation process; preference is given to using distillation or rectification.

Fluid connection between two regions or plant components is to be understood here as meaning any kind of connection that enables flow of a fluid, for example a reaction product or a hydrocarbon fraction, from one to the other of the two regions, regardless of any intermediately connected regions, components or required conveying means.

A means is to be understood as meaning something that enables or is helpful in the achievement of a goal. In particular, means for performing a particular process step are to be understood as including all physical articles that would be considered by a person skilled in the art in order to be able to perform this process step. For example, a person skilled in the art will consider means of introducing or discharging a material stream to include all transporting and conveying apparatuses, i.e. for example pipelines, pumps, compressors, valves, which seem necessary or sensible to said skilled person for performance of this process step on the basis of his knowledge of the art.

Oxygenates are in principle to be understood as meaning all oxygen-containing hydrocarbon compounds that can be converted under oxygenate conversion conditions to olefins, especially to short-chain olefins such as propylene, and further hydrocarbon products.

Short-chain olefins in the context of the present invention are especially understood as meaning olefins that are gaseous under ambient conditions, for example ethylene, propylene and the isomeric butenes 1-butene, cis-2-butene, trans-2-butene, isobutene.

Higher hydrocarbons in the context of the present invention are especially to be understood as meaning hydrocarbons that are liquid under ambient conditions.

Hydrocarbon fractions are identified using the following nomenclature: “Cn fraction” refers to a hydrocarbon fraction containing predominantly hydrocarbons of carbon chain length n, i.e. having n carbon atoms. “Cn−fraction” refers to a hydrocarbon fraction containing predominantly hydrocarbons of carbon chain length n but also containing shorter carbon chain lengths. “Cn+ fraction” refers to a hydrocarbon fraction containing predominantly hydrocarbons of carbon chain length n but also containing longer carbon chain lengths. Owing to the physical separation processes used, for example distillation, separation in terms of carbon chain length should not be considered to mean that hydrocarbons having another chain length are rigorously excluded. For instance, a Cn−fraction, depending on the process conditions of the separation process, will still contain small amounts of hydrocarbons having a carbon number greater than n.

The recited solid, liquid and gaseous/vaporous states of matter should always be considered in relation to the local physical conditions prevailing in the respective process step or in the respective plant component unless otherwise stated. In the context of the present application, the gaseous and vaporous states of matter should be considered to be synonymous. The term “vaporous” merely serves to illustrate that the particular substance is normally liquid under ambient conditions.

In the context of the present invention separating a material stream is to be understood as meaning division of the stream into at least two substreams. Unless otherwise stated it may be assumed that the physical composition of the substreams corresponds to that of the starting stream except in cases where it is immediately apparent to a person skilled in the art that there must inevitably be a change in the physical composition of the substreams owing to the separation conditions.

A gasoline fraction is to be understood as meaning a substance mixture which is in liquid form under ambient conditions, consists predominantly, preferably substantially completely, of higher hydrocarbons and may be suitable for use as a gasoline fuel.

The predominant portion of a fraction, of a material stream etc. is to be understood as meaning a proportion quantitatively greater than all other proportions each considered alone. Especially in the case of binary mixtures or in the case of separating a fraction into two parts this is to be understood as meaning a proportion of more than 50% by weight unless otherwise stated in the specific case.

The indication that a material stream consists predominantly of one component or group of components is to be understood as meaning that the mole fraction or mass fraction of this component or component group is quantitatively greater than all other proportions of other components or component groups in the material stream each considered alone. Especially in the case of binary mixtures this is to be understood as meaning a proportion of more than 50%. Unless otherwise stated in the specific case this is based on the mass fraction.

The indication that material streams are introduced into certain regions, for example the OTO synthesis reactor, exclusively in gaseous or vaporous form is to be understood as meaning that either no liquid constituents at all are present in the introduced material stream or that at most small proportions of ultrafine, gas-borne liquid droplets such as aerosols are present in the introduction stream. Furthermore, in the context of the invention this indication relates to the steady state of the process/of the plant. It cannot be ruled out that during transitional states such as for example during startup or shutdown of the plant condensation or liquid entrainment during time-limited operating periods may result in liquid proportions also passing into the reaction sections.

Pressure indications are in bar, absolute, bar(a) for short, unless otherwise stated in the particular context.

The invention is based on the recognition that the feeding and distribution system of the oxygenates-containing reactant mixture may be significantly simplified if all reactant mixture substreams, steam streams and recycle streams are introduced into the OTO synthesis reactor exclusively in gaseous/vaporous form. In terms of the reactant mixture sent from the DME reactor to the OTO reactor this is achieved when the entire DME reactor product is sent to the OTO reactor and applied thereto in gaseous form without a preceding cooling and/or partial condensation and separation into a DME-enriched gas phase and a DME-depleted liquid phase containing unconverted methanol and water that is separately sent to the OTO reactor and applied thereto. In this case the conventional vapour/liquid distribution system which typically contains two-phase nozzles is simplified to a simple gas distribution system without such nozzles. This reduces the pressure drop over the reactant mixture distribution system and compression energy is saved.

The distribution of a vapour side stream over a large cross sectional area is much easier than the atomization of liquid. Due to the low density of the vapour compared to a liquid a uniform distribution may be achieved with a pressure drop over the openings that is substantially lower than the pressure drop required for liquid distribution and atomization.

The total height of the reactor may be reduced since free length for evaporation of liquid droplets is no longer required inside the reaction sections. This reduces the construction costs of such a plant and the space required for erecting it.

The operating pressure of the oxygenate-containing feed stream sent to the OTO reactor upstream may be reduced since two-phase nozzles are not required. The two-phase nozzles in an OTO reactor of the prior art require a relatively high feed pressure on the upstream side both for feeding on the gas/vapour side and for feeding on the liquid side to promote atomization.

Operating the side feeding system at a lower pressure allows further cooling of the oxygenate-containing feed stream to the OTO reactor down to lower temperatures than before. The aim is to set the feeding temperature on the side of the oxygenate-containing vapour to a value slightly above the dew point so that the vapour pressure of the mixture at this operating temperature is always greater than the operating pressure.

Cooling to a lower temperature gives the oxygenate-containing feed stream a greater capacity for cooling the hot reaction product of the upstream reaction section.

Alternatively or in addition the cooling of the reaction sections may be achieved by supplying further cold gas streams to at least one, preferably two or more, most preferably all, reaction sections. One simple option is the use of process steam which is in any case available in an OTO plant since on the one hand it is produced as diluting steam and on the other hand water in vapour form is formed as a byproduct in the OTO reaction.

It is further advantageous when as a cold gas stream at least one hydrocarbon-containing material stream obtained in the fractionating workup of the OTO reactor product for example by fractionating distillation/rectification is recycled to the OTO reactor and therein supplied to at least one, preferably two or more, most preferably all, reaction sections. Light hydrocarbon recycle streams, in particular in the carbon number range C₂ to C₄, are preferred since they have a low dew point and can therefore be cooled to a particularly low temperature before addition to the reaction sections, thus in turn allowing particularly effective temperature control of the reaction sections. The prior art merely discloses applying such a light hydrocarbon stream as a recycle stream to only the first reaction section in the direction of flow.

It has surprisingly been found that a distribution of one or more light hydrocarbon streams as recycle streams over two or more, preferably all, reaction sections, results in improved reaction conditions due to reduced partial pressure of the reactant components in the individual catalyst beds. The reduced partial pressure of the reactants increases the selectivity of the OTO reaction toward desired target components such as in particular ethylene and propylene.

In addition, the light hydrocarbon stream(s) used as recycle streams may be premixed with the oxygenate-containing reactant mixture substreams sent to the reaction sections and/or the vapour streams sent to the reaction sections before they are applied to the reaction sections. This reduces the partial pressure and thus the dew point of the water/of the methanol in the mixed streams so that these streams too can be cooled to a greater extent before introduction into the individual reactions sections.

### BRIEF DESCRIPTION OF THE DRAWINGS

The invention is more particularly elucidated hereinbelow by way of an example without limiting the subject matter of the invention. Further features, advantages and possible applications of the invention will be apparent from the following description of the working example in conjunction with the drawings.

FIG. 1 shows a schematic diagram of an exemplary embodiment of the process according to the invention/the plant according to the invention,

FIG. 2 shows a schematic detailed diagram of the OTO synthesis reactor with the accompanying feed distribution system in an exemplary configuration.

### DETAILED DESCRIPTION OF THE INVENTION

A preferred embodiment of the process according to an embodiment of the invention is characterized in that all reaction sections are supplied with reactant mixture substreams on the one hand and with steam streams and/or recycle streams on the other hand. These measures ensure a particularly uniform distribution of the reactant components over the reaction sections, thus resulting in very good temperature control in the catalyst zones. In addition, the partial pressure of the reactants is uniformly kept at a low level which results in a reduced partial pressure of the reactant components in the individual catalyst zones. The reduced partial pressure of the reactants increases the selectivity of the OTO reaction toward desired target components such as in particular ethylene and propylene. The reactants in the abovementioned context include not only the oxygenates supplied to the reaction sections but also the hydrocarbons, in particular olefins, recycled to the reaction sections via the recycle streams which may likewise be converted into the abovementioned target components.

In a further preferred embodiment of the process according to the invention at least two hydrocarbon product fractions are recycled to the OTO synthesis reactor as recycle streams and introduced thereto. This allows the different properties of different hydrocarbon product fractions to be better utilized. Thus, hydrocarbon product fractions containing low molecular weight, low-boiling hydrocarbons are more suitable as gaseous coolant streams than higher molecular weight, higher-boiling hydrocarbons owing to their low dew point. On the other hand the latter have a higher potential as reactive components since especially higher molecular weight olefins having carbon numbers greater than four are particularly easily converted by catalytic cracking over the OTO synthesis catalyst into low molecular weight olefins such as ethylene and propylene in particular. Nevertheless the hydrocarbons in the product fractions containing low molecular weight, low-boiling hydrocarbons are also partially converted into low molecular weight olefins, albeit to a lesser extent than olefin-containing fractions having higher molecular weight, higher-boiling hydrocarbons.

It is particularly preferable when a hydrocarbon product fraction containing predominantly C₂ to C₈ hydrocarbons is introduced into the first reaction section as a recycle stream. This fraction has both good coolant properties and a high proportion of higher molecular weight components such as olefins as reactive components. Introducing this fraction into the first reaction section is particularly advantageous since this maximizes the residence time of this fraction in the OTO reactor, thus allowing a particularly extensive conversion of the reactive components into low molecular weight olefins such as ethylene and propylene in particular.

In a development of the two abovementioned particular embodiments of the process according to the invention exclusively a hydrocarbon product fraction containing predominantly C₂ to C₄ hydrocarbons is introduced into the at least one subsequent reaction section as recycle stream.

This utilizes the good coolant properties of the low molecular weight, low-boiling hydrocarbons particularly effectively while simultaneously maximizing the residence time of the hydrocarbon product fraction containing predominantly C₂ to C₈ hydrocarbons in the OTO reactor, thus allowing a particularly extensive conversion of the reactive components into low molecular weight olefins such as ethylene and propylene in particular.

A further, preferred embodiment of the process according to the invention provides that the pressure drop over a feeding apparatus for a reactant mixture substream is less than 5 bar(a), preferably less than 3 bar(a). Operating experience and further investigations have shown that these pressure drops are markedly below those occurring when adding liquid/gaseous oxygenate mixtures using two-fluid nozzles in prior art processes. This makes it possible to use lower pressures in the DME reactor and the equipment parts arranged upstream thereof, thus allowing a more cost-effective design.

In a further aspect the process according to the invention is characterized in that the mass flow of the recycle stream and/or the mass flow of the steam is separately controlled or regulated for at least two reaction sections. Particularly flexible operation of the OTO synthesis reactor is thereby made possible and thermal fluctuations may be readily compensated.

A further preferred embodiment of the process according to the invention is characterized in that the oxygenate partial pressure inside the catalyst stage is between 0.1 and 0.5 bar(a). Investigations have shown that establishing these oxygenate partial pressures results in a particularly advantageous reactor productivity since this achieves a compromise between a high selectivity for short-chain olefins such as ethylene and propylene on the one hand (favoured by lowest possible oxygenate partial pressure) and a high oxygenate throughput on the other hand (favoured by highest possible oxygenate partial pressure).

In a further aspect the process according to the invention is characterized in that the oxygenates-containing reactant mixture contains dimethyl ether (DME) and is produced in an etherification reactor by catalytic dehydration of methanol in the gas phase to obtain a gaseous etherification reactor product mixture comprising DME, steam and methanol vapour, wherein the gaseous etherification reactor product mixture is sent to the OTO synthesis reactor as reactant mixture without an additional separation step. It is advantageous when there is no separation of the etherification reactor product mixture into a gas phase and a liquid phase that must be sent to the OTO synthesis reactor and applied thereto separately. This saves cooling energy, a corresponding separation apparatus is omitted and the conduit system is simplified.

In a further aspect the process according to the invention is characterized in that the oxygenates-containing reactant mixture contains dimethyl ether (DME) and is produced in an etherification reactor by catalytic dehydration of methanol in the gas phase to obtain a gaseous etherification reactor product mixture comprising DME, steam and methanol vapour, wherein the gaseous etherification reactor product mixture is sent to the OTO synthesis reactor as a reactant mixture without an additional separation step and wherein the oxygenates-containing reactant mixture has a DME content between 50% and 70% by weight, preferably between 55% and 60% by weight. Investigations have shown that these oxygenate contents in the reactant mixture may be particularly readily processed in the downstream OTO synthesis reactor.

In a further aspect the process according to the invention is characterized in that the oxygenates-containing reactant mixture contains dimethyl ether (DME) and is produced in an etherification reactor by catalytic dehydration of methanol in the gas phase to obtain a gaseous etherification reactor product mixture comprising DME, steam and methanol vapour, wherein the gaseous etherification reactor product mixture is sent to the OTO synthesis reactor as a reactant mixture without an additional separation step and wherein the absolute pressure of the oxygenates-containing reactant mixture before introduction into the OTO synthesis reactor is less than 7 bar(a), preferably less than 6 bar(a), and the temperature of the oxygenates-containing reactant mixture is set such that it is at least 5° C., preferably at least 10° C., above the dew point at this pressure. Investigations have shown that these process conditions allow long-lasting, stable operation of the process without premature catalyst deactivation and while simultaneously achieving a good yield of low molecular weight olefins such as ethylene and propylene in particular.

In a further aspect the process according to the invention is characterized in that the oxygenates-containing reactant mixture contains dimethyl ether (DME) and is produced in an etherification reactor by catalytic dehydration of methanol in the gas phase to obtain a gaseous etherification reactor product mixture comprising DME, steam and methanol vapour, wherein the gaseous etherification reactor product mixture is sent to the OTO synthesis reactor as a reactant mixture without an additional separation step and wherein the absolute pressure of the oxygenates-containing reactant mixture before introduction into the OTO synthesis reactor is less than 7 bar(a), preferably less than 6 bar(a), and the temperature of the oxygenates-containing reactant mixture is at least 140° C., preferably at least 150° C. Investigations have shown that these process conditions allow particularly long-lasting, stable operation of the process without premature catalyst deactivation and while simultaneously achieving a very good yield of low molecular weight olefins such as ethylene and propylene in particular.

In a further aspect the process according to the invention is characterized in that a first reaction section and five subsequent reaction sections are present.

In a further aspect the process according to the invention is characterized in that the conversion in the OTO synthesis reactor is carried out at temperatures of 300° C. to 600° C., preferably at temperatures of 360° C. to 550° C., most preferably at temperatures of 400° C. to 500° C.

In a further aspect the process according to the invention is characterized in that the conversion in the OTO synthesis reactor is carried out at pressures of 0.1 to 20 bar, absolute, preferably at pressures of 0.5 to 5 bar, absolute, most preferably at pressures of 1 to 3 bar, absolute.

In a further aspect the process according to the invention is characterized in that the catalyst zones in the reaction sections contain a granular, shape-selective zeolite catalyst of the pentasil type, preferably ZSM-5, in the form of a fixed bed.

In a particular aspect of the plant according to the invention all reaction sections are provided with means for introducing reactant mixture substreams on the one hand and with means for introducing steam streams and/or recycle streams on the other hand. These constructional features ensure a particularly uniform distribution of the reactant components over the reaction sections, thus resulting in very good temperature control in the catalyst zones. In addition, the partial pressure of the reactants is uniformly kept at a low level which results in a reduced partial pressure of the reactant components in the individual catalyst zones. The reduced partial pressure of the reactants increases the selectivity of the OTO reaction toward desired target components such as in particular ethylene and propylene. The reactants in the abovementioned context include not only the oxygenates supplied to the reaction sections but also the hydrocarbons, in particular olefins, recycled to the reaction sections via the recycle streams which may likewise be converted into the abovementioned target components.

It is preferable when the plant according to the invention comprises a workup apparatus having a plurality of separation stages in which different hydrocarbon fractions are obtained and further comprises at least two recycle conduits which recycle from different separation stages to the OTO synthesis reactor and which are connected to different means for introducing recycle streams into the reaction sections. This makes it possible for at least two hydrocarbon product fractions to be recycled to the OTO synthesis reactor as recycle streams and introduced thereto. This allows the different properties of different hydrocarbon product fractions to be better utilized. Thus, hydrocarbon product fractions containing low molecular weight, low-boiling hydrocarbons are more suitable as gaseous coolant streams than higher molecular weight, higher-boiling hydrocarbons owing to their low dew point. On the other hand the latter have a higher potential as reactive components since especially higher molecular weight olefins having carbon numbers greater than four are particularly easily converted by catalytic cracking over the OTO synthesis catalyst into low molecular weight olefins such as ethylene and propylene in particular. Nevertheless the hydrocarbons in the product fractions containing low molecular weight, low-boiling hydrocarbons are also partially converted into low molecular weight olefins, albeit to a lesser extent than olefin-containing fractions having higher molecular weight, higher-boiling hydrocarbons.

In the finally elucidated embodiment it is particularly preferable when a first separation stage is connected to the first reaction section via a first recycle conduit and when a second separation stage is connected to at least one subsequent reaction section via a second recycle conduit. This makes it possible in particular for a hydrocarbon product fraction containing predominantly C₂ to C₈ hydrocarbons to be introduced into the first reaction section via the first recycle conduit as recycle stream and for exclusively a hydrocarbon product fraction containing predominantly C₂ to C₄ hydrocarbons to be introduced into the at least one subsequent reaction section via the second recycle conduit as recycle stream. This utilizes the good coolant properties of the low molecular weight, low-boiling hydrocarbons in the carbon number range C₂ to C₄ particularly effectively while simultaneously maximizing the residence time of the hydrocarbon product fraction containing predominantly C₂ to C₈ hydrocarbons in the OTO reactor, thus allowing a particularly extensive conversion of the reactive components into low molecular weight olefins such as ethylene and propylene in particular.

A further aspect of the plant according to the invention is characterized in that it further comprises an etherification reactor arranged upstream of the OTO synthesis reactor which is configured such that by catalytic dehydration of methanol in the gas phase a gaseous, oxygenates-containing reactant mixture that can be sent to the OTO synthesis reactor without an additional separation step is obtainable. It is advantageous when there is no separation of the etherification reactor product mixture into a gas phase and a liquid phase that must be sent to the OTO synthesis reactor and applied thereto separately. This saves cooling energy, a corresponding separation apparatus is omitted and the conduit system is simplified.

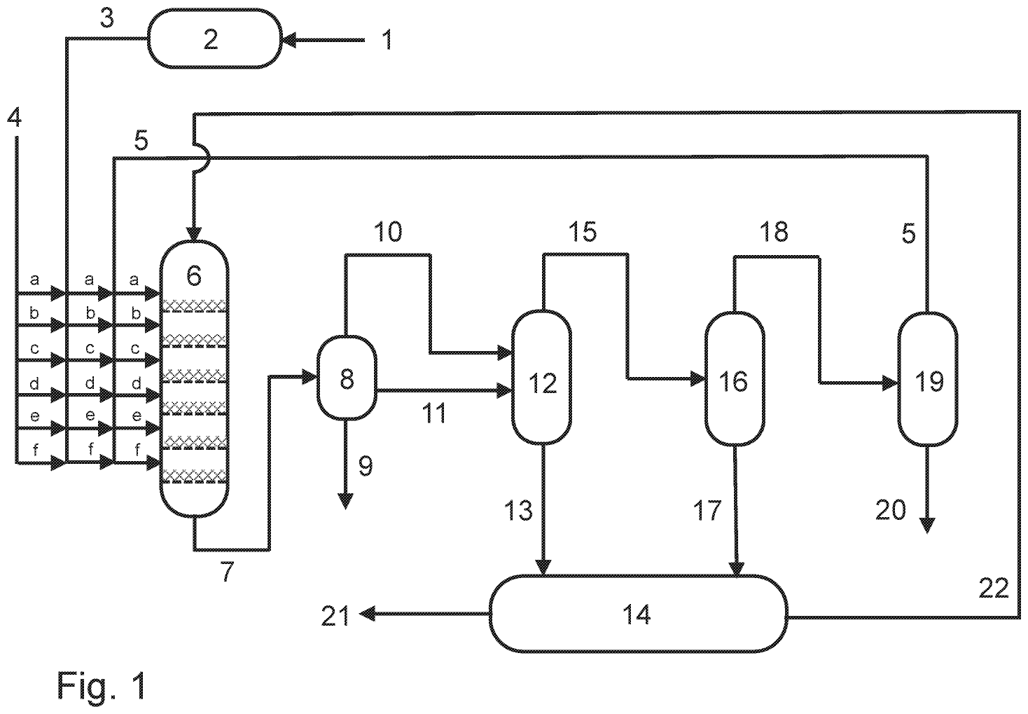

FIG. 1 shows a schematic diagram of an exemplary embodiment of the process according to the invention/the plant according to the invention for producing an olefins-containing hydrocarbon product comprising in particular the short-chain olefins ethylene and propylene as value products by conversion of an oxygenates-containing reactant mixture. To produce the oxygenates-containing reactant mixture initially methanol vapour, optionally in conjunction with steam as diluent, is applied via conduit 1 to the dehydration reactor (DME reactor) 2 which has been filled with a dumped fixed bed of a commercially available dehydration catalyst. Effected over this catalyst is a heterogeneously catalysed partial conversion of the methanol to dimethyl ether (DME) under dehydration conditions known to those skilled in the art.

In certain embodiments of the present invention, the obtained gaseous product mixture from the dehydration reactor, which comprises not only DME but also unconverted methanol and steam, can be applied without cooling and phase separation but rather still in gaseous form by means of conduit 3 directly to the OTO synthesis reactor 6, which in the present case comprises six reaction sections. Division into six reactant mixture substreams and distribution thereof to the six reaction sections is carried out using the conduit system 3a to 3f. In addition, via the conduit system 4a to 4f steam may be supplied and likewise distributed over the six reaction sections. Finally, via the conduit system 5a to 5f a gas stream containing predominantly C₂ hydrocarbons is recycled to the OTO synthesis reactor and distributed over the six reaction sections. The gas distributor system shown in FIG. 1 is to be understood as being purely schematic. In particular embodiments the individual gas types—reactant mixture, steam, hydrocarbon recycle stream—may be applied to the reaction sections either separately or premixed. Premixing of the gas streams is preferable since this reduces the partial pressure of the reactive components, thus resulting in improved temperature management of the OTO synthesis reactor and improved selectivity for short-chain olefins. Possible operating modes of the reactor include those in which said reactor is supplied either with oxygenate-containing reactant mixture and steam as diluent or with oxygenate-containing reactant mixture and a hydrocarbon recycle stream as diluent or with oxygenate-containing reactant mixture and both steam and a hydrocarbon recycle stream as diluent. The latter operating mode is preferred especially when the steam content in conduit 3 and the amount of the hydrocarbon recycle stream are not yet sufficient to allow adequate temperature control and partial pressure adjustment in the reaction sections. It provides the greatest flexibility among the elucidated operating modes.

It is also possible as a particular embodiment of the invention to supply steam and hydrocarbon to one or more reaction sections, the oxygenate content being reduced to zero in extreme cases save for a small value. This optimizes the conversion of specific recycle streams or else hydrocarbon-containing streams from other processes may be incorporated. It is especially preferred when these reactant mixture substreams are added to the downstream reaction sections of the OTO reactor, particularly preferably to the last reaction section, having an oxygenate content that has been reduced or reduced to zero.

Supply of the first reaction section with C₂ hydrocarbons via the conduit path 5a may optionally also be omitted since the first reaction section is already being supplied with a hydrocarbon recycle stream via conduit 22.

The conversion of the oxygenates and hydrocarbon reactive components in the reaction sections of the OTO synthesis reactor is effected under oxygenate conversion conditions known to those skilled in the art and disclosed in the relevant literature. To this end the reaction sections are provided with catalyst zones provided with fixed dumped beds of a commercially available olefin synthesis catalyst.

The product mixture of the OTO synthesis reactor is discharged therefrom via conduit 7 and supplied to the multistage product workup which is shown in FIG. 1 merely in highly schematic form and is subsequently elucidated only to the extent required for understanding the present invention. Initially carried out in quench stage 8 is a cooling of the product mixture below the dew point and subsequently a phase separation into an aqueous phase discharged via conduit 9 as well as into a gaseous phase and into a liquid phase which each contain predominantly hydrocarbons, are discharged via conduits 10 and 11 from the quench stage and are both applied to a distillation column 12 known as a debutanizer.

The debutanizer distillation column 12 separates the hydrocarbon stream supplied via conduits 10 and 11 by fractionating distillation. Discharged from the column 12 as the bottoms product is a hydrocarbon fraction containing hydrocarbons having four or more carbon atoms (C₄₊ fraction). Said fraction is supplied via conduit 13 to a workup apparatus for heavy hydrocarbon fractions 14. The further separation of the hydrocarbon mixture is carried out therein by means of a plurality of separating operations, for example multistage distillation, extraction, extractive distillation.

The tops product from the column 12 forms a hydrocarbon fraction containing hydrocarbons having four or less carbon atoms (C₄₋ fraction). This fraction also contains hitherto unconverted oxygenates. It is discharged from column 12 via conduit 15 and applied to a distillation column 16 known as a depropanizer.

The depropanizer distillation column 16 separates the hydrocarbon stream supplied via conduit 15 by fractionating distillation. Discharged from the column 16 as the bottoms product is a hydrocarbon fraction containing hydrocarbons having four carbon atoms and unconverted oxygenates (C₄O fraction). Said fraction is supplied via conduit 17 to the workup apparatus for heavy hydrocarbon fractions 14. The further separation of the hydrocarbon mixture is carried out therein by means of a plurality of separating operations, for example multistage distillation, extraction, extractive distillation.

The tops product from the column 16 forms a hydrocarbon fraction containing hydrocarbons having three or less carbon atoms (C₃₋ fraction). It is discharged from column 16 via conduit 18 and applied to a distillation column 19 known as a deethanizer.

The deethanizer distillation column 19 separates the hydrocarbon stream supplied via conduit 18 by fractionating distillation. Discharged from the column 19 as a bottoms product is a hydrocarbon fraction which comprises hydrocarbons having three carbon atoms and thus comprises not only propane but also the target product propylene. It is supplied via conduit 20 to a workup apparatus (not shown) in which propane and propylene are separated by distillation and which contains optionally further workup stages so that the target product propylene is obtainable in pure form.

The tops product from the column 19 forms a hydrocarbon fraction containing hydrocarbons having two or less carbon atoms (C₂₋ fraction). It is discharged from column 19 via conduit 5 and after further optional workup or conditioning steps (not shown) is separated into a substream which is discharged from the process as a purge stream via a conduit (not shown). If desired, ethylene may also be obtained from the purge stream as a pure product by workup steps that are known per se. From the remaining proportion a smaller substream is removed as purge and the remaining stream of the C₂₋ fraction is recycled to the OTO synthesis reactor via conduit 5.

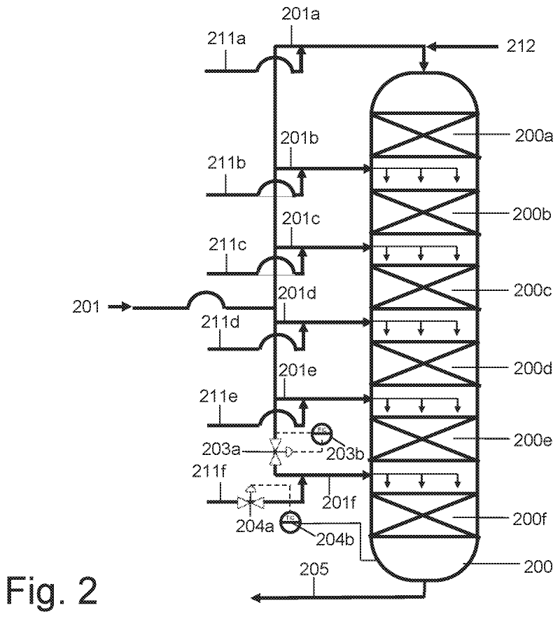

The OTO synthesis reactor 200 shown schematically in FIG. 2 for conversion of DME into olefins is in the form of a fixed bed reactor having a plurality of reaction sections 200a-200f which each contain zones of a catalyst reactive and selective for OTO synthesis. It is advantageous to provide at least three, preferably at least four, most preferably, as shown in FIG. 2, six, catalyst stages. This embodiment of the OTO synthesis reactor is an advantageous compromise. Yet more reaction sections would further reduce the reaction enthalpy liberated per section and would therefore be advantageous for temperature control of the reactor; however the increasing capital costs and increasing control complexity would be disadvantageous.

Supplying with dimethyl ether as the oxygenate is carried out by dividing the reactant stream in conduit 201 into the individual reactant substreams in conduits 201a to 201f Simultaneously via conduits 211a to 211f all reaction sections are supplied with a C₂ hydrocarbons-containing recycle gas; as elucidated with reference to FIG. 1 this may be a substream of the tops product from the deethanizer. Furthermore, via conduit 212 the first reaction sections are supplied with a C₄ to C₆ hydrocarbons-containing recycle gas obtained by working up the bottoms products from the debutanizer and the depropanizer. The latter may also contain proportions of unconverted DME which are likewise recycled to the OTO synthesis reactor. All of the streams applied to the reactor 200 may be combined also with steam; alternatively or in addition steam may be added to one or more reaction sections via feed conduits (not shown). This is advantageous especially when the steam stream is to be controlled separately from the reactant substreams or recycle streams for improved temperature control. It is essential and characterizing to the invention that all of these material streams are applied to the OTO synthesis reactor in gaseous form. This may be achieved for example by choosing the temperature for the C2 hydrocarbons-containing recycle gas of between 0° C. and 50° C. and for the steam of between 100° C. and 220° C. Due to the proportion of higher-boiling hydrocarbons the temperature of the C4- to C6-hydrocarbons-containing recycle gas must be higher than that of the first recycle gas; it is essential that the temperature is safely above the dew point which depends on the precise composition of the fraction.

The individual reaction sections are arranged in series. By mixing the cold input gas with the hot product gas exiting the preceding catalyst stage the latter is cooled and may therefore react in the desired temperature range with the admixed dimethyl ether and the reactive components in the recycle gas in the subsequent reaction stage.

Mixing of a dimethyl ether-containing reactant substream and recycle gas is shown exemplarily in the last stage 200f. A flow controller 203b and the control valve 203a assigned thereto are used to adjust the reactant substream such that the desired oxygenate amount is introduced into the reaction section 200f. The cold reactant substream supplied via conduit 201f does already achieve a certain cooling when this stream mixes with the product stream from the upstream reaction section 200e. In addition, C2-containing recycle gas and/or steam may be added via valve 204a so that via the temperature controller 204b the desired target temperature of the exit stream from the reaction section is also achieved.

This temperature and reaction management concept is advantageously implemented in the same way for all other reaction sections but at least for the reaction sections 2 to 6. The entry and exit temperatures for the respective stage are flexible and easily adjustable via the quantity ratio of the respective DME and recycling streams. It is thus possible to establish over the entire reactor a temperature profile optimal for a maximum ethylene and/or propylene yield.

### Numerical Examples

Specifically a reactor as shown in FIG. 2 may be advantageously operated with the following settings:

A preselected temperature level may be established over the reaction sections 200a and 200e of the reactor and in the next reaction section additional cooling with oxygenate, a recycle gas consisting predominantly of C₂ hydrocarbons having a preferred temperature between 120° C. and 160° C. and/or process steam may be minimized. The temperatures in the reaction sections 200a and 200e are preferably between 470° C. and 500° C. All of the material streams added to the reaction sections are gaseous and were measured such that per reaction section virtually the same temperature increase is obtained as in a process according to the prior art with the same six-stage reactor but biphasic supply of the reactant mixture in gas/liquid form via two-fluid nozzles.

In the last reaction section 200f a reduced conversion of DME/a largely flat temperature interval is established over the reaction section. According to the invention the temperature profile in the reaction section 200f then varies for example between 480° C. and 500° C. Thus at maximum temperature and low reformation from DME a very largely complete reaction of the C₂ to C₄ olefins present in the reaction gas to afford propylene is achieved. Comparable settings are possible in a prior art configuration of the reactor only to a limited extent since the exothermicity of the corresponding reaction in the presence of oxygenates requires low entry temperatures.

Cooling the oxygenate and recycle gas streams to 120° C. to 160° C. results in efficient cooling of the product gas upon introduction of the oxygenate recycle gas mixture into the reaction sections. In example 1 reported hereinbelow in table 1 the use of about 84% by weight of ethylene in the recycle gas and without steam introduction results in the process data summarized therein with regard to cooling in the individual reaction sections.

In place of the above-described recycle gas stream cooling may also be achieved by admixing process steam with the oxygenate stream via a separate side feed before application to the respective reaction section as reported hereinbelow in table 2 as example 2.

A further option in a further embodiment of the invention is that of combining a recycle gas stream and a process steam stream with an oxygenate stream and applying them to a reaction section together as shown hereinbelow in table 3 as example 3.

TABLE 1
Cooling of individual reaction sections
using DME and C₂ recycle gas (Example 1)

| | Cooling demand | Cooling | Cooling by |
|---|---|---|---|
| | upstream of | by DME | C₂ recycle |
| | section | (gas) | gas |
| Reaction section #1 | 0% | — | — |
| Reaction section #2 | 100% | 95.3% | 4.7% |
| Reaction section #3 | 100% | 88.9% | 11.1% |
| Reaction section #4 | 100% | 84.0% | 16.0% |
| Reaction section #5 | 100% | 80.4% | 19.6% |
| Reaction section #6 | 100% | 77.6% | 22.4% |

TABLE 2
Cooling of individual reaction section
using DME and process steam (Example 2)

| | Cooling demand | Cooling | Cooling by |
|---|---|---|---|
| | upstream of | by DME | process |
| | section | (gas) | steam |
| Reaction section #1 | 0% | — | — |
| Reaction section #2 | 100% | 89.0% | 11.0% |
| Reaction section #3 | 100% | 84.7% | 15.3% |
| Reaction section #4 | 100% | 81.8% | 18.2% |
| Reaction section #5 | 100% | 79.7% | 20.3% |
| Reaction section #6 | 100% | 78.3% | 21.7% |

The lower entry temperatures also reduce the partial pressures of the individual reactants as summarized for the above three examples and compared with a prior art embodiment hereinbelow in table 4.

Under otherwise comparable conditions an OTO plant based on a gaseous DME reactant stream and gaseous diluents can achieve an up to 2% higher propylene selectivity than a comparative plant according to the prior art. The selectivity increase is achieved due to the abovementioned reduction in the partial pressure of the reactive components and also due to the fact that the reaction temperatures can be kept in the optimal range in the individual reaction sections by the temperature management according to the invention.

TABLE 3
Cooling of individual reaction section using DME,
C₂-recycle gas and process steam (Example 3)

| | Cooling demand | Cooling | Cooling by | Cooling by |
|---|---|---|---|---|
| | upstream of | by DME | C₂ recycle | process |
| | section | (gas) | gas | steam |
| Reaction | 0% | — | — | — |
| section #1 | | | | |
| Reaction | 100% | 87.3% | 5.9% | 6.8% |
| section #2 | | | | |
| Reaction | 100% | 81.2% | 12.4% | 6.4% |
| section #3 | | | | |
| Reaction | 100% | 76.5% | 17.4% | 6.1% |
| section #4 | | | | |
| Reaction | 100% | 73.0% | 21.1% | 5.9% |
| section #5 | | | | |
| Reaction | 100% | 70.3% | 23.9% | 5.7% |
| section #6 | | | | |

TABLE 4
Partial pressures of the reactants upon entry and exit for
individual reaction sections (all pressures in bar(a)).

| | Compar- | | | |
|---|---|---|---|---|
| P_{React} = reactants | ative ex. | Example 1 | Example 2 | Example 3 |
| partial pressure | (prior art) | (invention) | (invention) | (invention) |
| P_{React} to | 0.438 bar | 0.320 bar | 0.486 bar | 0.322 bar |
| section #1 | | | | |
| P_{React} from | 0.357 bar | 0.250 bar | 0.395 bar | 0.251 bar |
| section #1 | | | | |
| P_{React} to | 0.410 bar | 0.318 bar | 0.446 bar | 0.319 bar |
| section #2 | | | | |
| P_{React} from | 0.331 bar | 0.249 bar | 0.359 bar | 0.248 bar |
| section #2 | | | | |
| P_{React} to | 0.381 bar | 0.317 bar | 0.404 bar | 0.317 bar |
| section #3 | | | | |
| P_{React} from | 0.303 bar | 0.246 bar | 0.321 bar | 0.245 bar |
| section #3 | | | | |
| P_{React} to | 0.351 bar | 0.315 bar | 0.363 bar | 0.312 bar |
| section #4 | | | | |
| P_{React} from | 0.274 bar | 0.241 bar | 0.283 bar | 0.239 bar |
| section #4 | | | | |
| P_{React} to | 0.321 bar | 0.309 bar | 0.322 bar | 0.305 bar |
| section #5 | | | | |
| P_{React} from | 0.245 bar | 0.234 bar | 0.246 bar | 0.231 bar |
| section #5 | | | | |
| P_{React} to | 0.292 bar | 0.284 bar | 0.302 bar | 0.297 bar |
| section #6 | | | | |
| P_{React} from | 0.216 bar | 0.210 bar | 0.224 bar | 0.220 bar |
| section #6 | | | | |

### LIST OF REFERENCE NUMERALS

1 conduit
2 DME reactor
3-5 conduit
6 OTO synthesis reactor
7 conduit
8 quench
9-11 conduit
12 separating column (debutanizer)
13 conduit
14 workup apparatus
15 conduit
16 separating column (depropanizer)
17-18 conduit
19 separating column (deethanizer)
20-22 conduit
200 OTO synthesis reactor
201 conduit
203a, 204a control valve
203b flow meter
204b temperature measurement
205 conduit
211-212 conduit

1. A process for producing an olefins-containing hydrocarbon product comprising ethylene and propylene by conversion of an oxygenates-containing reactant mixture, which is divided into a plurality of reactant mixture substreams, in a multi-stage oxygenate-to-olefin (OTO) synthesis reactor, the process comprising the following steps:
(a) providing the multistage OTO synthesis reactor having a plurality of serially connected reaction sections in fluid connection with one another comprising a first reaction section and at least one subsequent reaction section which each contain catalyst zones comprising solid catalysts that are active and selective for OTO synthesis, wherein upstream of each catalyst zone a feeding apparatus for a reactant mixture substream is arranged and wherein the last reaction section in the direction of flow is in fluid connection with a conduit for discharging an OTO synthesis reactor product;
(b) introducing a reactant mixture substream into each reaction section via the respective feeding apparatus, wherein the at least one subsequent reaction section is additionally supplied with the product stream from the respective upstream reaction section, introducing at least one steam stream into at least one reaction section, introducing at least one recycle stream into at least one reaction section;
(c) at least partially converting the supplied oxygenates in the catalyst zones under oxygenate conversion conditions into olefins and further hydrocarbons, discharging the OTO synthesis reactor product,
(d) separating the OTO synthesis reactor product in a multistage workup apparatus operating by means of thermal separation processes to obtain a plurality of hydrocarbons-containing hydrocarbon product fractions,
(e) discharging an olefins-containing, in particular ethylene- and/or propylene-containing, hydrocarbon product from the workup apparatus,
(f) recycling at least a portion of one or more hydrocarbon product fractions to the OTO synthesis reactor as a recycle stream or recycle streams and introducing the recycle stream(s) into at least one reaction section,
wherein all reactant mixture substreams, steam streams, and recycle streams are introduced into the OTO synthesis reactor exclusively in gaseous/vaporous form.

2. The process according to claim 1, wherein all reaction sections are supplied with reactant mixture substreams on the one hand and with steam streams and/or recycle streams on the other hand.

3. The process according to claim 1, wherein at least two hydrocarbon product fractions are recycled to the OTO synthesis reactor as recycle streams and introduced thereto.

4. The process according to claim 1, wherein a hydrocarbon product fraction containing predominantly C₂ to C₈ hydrocarbons is introduced into the first reaction section as a recycle stream.

5. The process according to claim 1, wherein exclusively a hydrocarbon product fraction containing predominantly C₂ to C₄ hydrocarbons is introduced into the at least one subsequent section as a recycle stream.

6. The process according to claim 1, wherein the pressure drop over a feeding apparatus for a reactant mixture substream is less than 5 bar(a), preferably less than 3 bar(a).

7. The process according to claim 1, wherein the mass flow of the recycle stream and/or the mass flow of the steam is separately controlled or regulated for at least two reaction sections.

8. The process according to claim 1, wherein the oxygenate partial pressure inside the catalyst stage is between 0.1 and 0.5 bar(a).

9. The process according to claim 1, wherein the oxygenates-containing reactant mixture contains dimethyl ether (DME) and is produced in an etherification reactor by catalytic dehydration of methanol in the gas phase to obtain a gaseous etherification reactor product mixture comprising DME, steam and methanol vapour, wherein the gaseous etherification reactor product mixture is sent to the OTO synthesis reactor as reactant mixture without an additional separation step.

10. The process according to claim 9, wherein the oxygenates-containing reactant mixture has a DME content between 50% and 70% by weight, preferably between 55% and 60% by weight.

11. The process according to claim 9, wherein the absolute pressure of the oxygenates-containing reaction mixture before introduction into the OTO synthesis reactor is less than 7 bar(a), preferably less than 6 bar(a), and the temperature of the oxygenates-containing reaction mixture is set such that the temperature is at least 5° C., preferably at least 10° C., above the dew point at this pressure.

12. The process according to claim 9, wherein the absolute pressure of the oxygenates-containing reaction mixture before introduction into the OTO synthesis reactor is less than 7 bar(a), preferably less than 6 bar(a), and the temperature of the oxygenates-containing reaction mixture is at least 140° C., preferably at least 150° C.

13. A plant for producing an olefins-containing hydrocarbon product comprising ethylene and propylene by conversion of an oxygenates-containing reactant mixture, which is divided into a plurality of reactant mixture substreams, in a multi-stage oxygenate-to-olefin (OTO) synthesis reactor comprising the following constituents:
(a) a multistage OTO synthesis reactor having a plurality of serially connected reaction sections in fluid connection with one another comprising a first reaction section and at least one subsequent reaction section which each contain catalyst zones comprising solid catalysts that are active and selective for OTO synthesis, wherein upstream of each catalyst zone a feeding apparatus for a reactant mixture substream is arranged and wherein the last reaction section in the direction of flow is in fluid connection with a conduit for discharging an OTO synthesis reactor product;
(b) means for introducing a reactant mixture substream into each reaction section via the respective feeding apparatus, means for introducing at least one steam stream into at least one reaction section, means for introducing at least one recycle stream into at least one reaction section,
(c) means for adjusting oxygenate conversion conditions, means for discharging the OTO synthesis reactor product,
(d) a multi-stage workup apparatus operating by means of thermal separation processes and suitable for separating the OTO synthesis reactor product into a plurality of hydrocarbons-containing hydrocarbon product fractions, means for introducing the OTO synthesis reactor product into the workup apparatus,
(e) means for discharging an olefins-containing, in particular ethylene- and/or propylene-containing, hydrocarbon product from the workup apparatus,
(f) means for recycling at least a portion of one or more hydrocarbon product fractions obtained in the workup apparatus to the OTO synthesis reactor as a recycle stream or recycle streams and means for introducing the recycle stream(s) into at least one reaction section,
wherein all means recited under (b) are configured such that all reactant mixture substreams, steam streams and recycle streams are introduceable into the OTO synthesis reactor in gaseous/vaporous form.

14. The plant according to claim 13, wherein all reaction sections are provided with means for introducing reactant mixture substreams on the one hand and with means for introducing steam streams and/or recycle streams on the other hand.

15. The plant according to claim 13, wherein the workup apparatus comprises a plurality of separation stages in which different hydrocarbon fractions are obtained and further comprises at least two recycle conduits which recycle from different separation stages to the OTO synthesis reactor and which are connected to different means for introducing recycle streams into the reaction sections.

16. The plant according to claim 15, wherein a first separation stage is connected to the first reaction section via a first recycle conduit and in that a second separation stage is connected to at least one subsequent reaction section via a second recycle conduit.

17. The plant according to claim 13, wherein it further comprises an etherification reactor arranged upstream of the OTO synthesis reactor which is configured such that by catalytic dehydration of methanol in the gas phase a gaseous, oxygenate-containing reactant mixture that can be sent to the OTO synthesis reactor without an additional separation step is obtainable.
